(19) United States
(12) Patent Application Publication
(10) Pub. No.: US 2009/0036840 A1
(43) Pub. Date: Feb. 5, 2009

(54) Atraumatic ball tip and side wall opening

(75) Inventors: Victor E. Viray, Sunnyvale, CA (US); David Callaghan, Mansfield, MA (US)

Correspondence Address: BINGHAM MCCUTCHEN LLP, 2020 K Street, N.W., Intellectual Property Department, WASHINGTON, DC, 20006, US

(73) Assignee: Cytyc Corporation

(21) Appl. No.: 12/232,842

(22) Filed: Sep. 25, 2008

Related U.S. Application Data
(63) Continuation-in-part of application No. 11/562,882, filed on Nov. 22, 2006.

(51) Int. Cl. A61M 25/00

(52) U.S. Cl. 604/264

(57) ABSTRACT

A delivery catheter with a plug ejection mechanism with a fluid filled actuator incorporated in the catheter handle is disclosed. After delivery of RF energy, the plug is deployed within the region of the lesion by activating the plug ejection mechanism. A delivery catheter used for female sterilization with an atraumatic ball tip and an opening in the side wall of the catheter body is disclosed. The delivery catheter includes a hinge that will yield or bend under a certain load to prevent injury to the uterine wall or fallopian tube. The side wall opening is angled to allow proper placement of a plug into the fallopian tube for occlusion of the fallopian tube.

### CROSS-REFERENCES

The present application is a continuation-in-part of U.S. patent application Ser. No. 11/562,882 filed Nov. 22, 2006 entitled “Delivery Catheter with Implant Ejection Mechanism” currently pending.

### FIELD OF THE INVENTION

The inventions described below relates to a system and method for implanting devices in the fallopian tubes or other vessels of the body.

### BACKGROUND OF THE INVENTION

In our prior U.S. patent, Harrington, et al., Flexible Method and Apparatus for Tubal Occlusion, U.S. Pat. No. 6,309,384 (Oct. 30, 2001), we described devices and methods for sterilization of female patients. Our sterilization method involves thermally wounding a small area of the patient's utero-tubal junction with relatively low power, and placing a foam plug within the wounded area. The method is facilitated by our catheter system, which comprises a catheter with a wounding segment which fits into the utero-tubal junction and carries the plug. The wounding segment comprises a short tubular extension slidably mounted within the distal tip of the catheter. The foam plug is stored within the wounding segment. The plug is deposited in the ovarian pathway when the wounding segment is retracted over the plug (a stationary holding rod within the catheter holds the plug in place relative to the catheter, so that retraction of the wounding segment exposes the plug).

### SUMMARY

In one aspect of the present invention, the systems and methods described below provide for smooth ejection or release of a contraceptive plug or other implant in a system requiring retraction of a sheath to eject or release the implant. In one embodiment, a plug ejection mechanism is incorporated into the catheter system to retract the sheath within a catheter body while holding the plug in place, thereby exposing the plug. The plug ejection mechanism comprises the sheath, a push rod inside the sheath, and a sheath retraction mechanism which includes a dashpot with a fluid filled chamber and a piston, a pre-loaded spring operably fixed to the sheath, and a latch that prevents any motion of the components until the mechanism is unlatched by the user. A push-button or solenoid-operated unlatching mechanism is provided to release the latch, thereby releasing the spring in the dashpot, thereby drawing the catheter sheath proximally. The fluid-filled chamber of the dashpot dampens the spring action to provide smooth and whip-less ejection of the plug from the sheath.

In another aspect of the present invention, a delivery catheter used for female sterilization with an atraumatic ball tip and an opening in the catheter side wall is disclosed. The delivery catheter includes a hinge that will yield or bend under a certain load to prevent injury to the uterine wall or fallopian tube. The side wall opening is angled to allow proper placement of a plug into the fallopian tube for occlusion of the fallopian tube.

### BRIEF DESCRIPTION OF THE DRAWINGS

FIG. 1 shows a sectional view of the delivery catheter illustrating the main components of the delivery catheter.

FIG. 2 shows the distal portion of the delivery catheter.

FIG. 3 shows a perspective view of the major components of the sheath retraction mechanism.

FIG. 4 illustrates the delivery catheter with the plug ejection mechanism as it is configured prior to ejection of the plug.

FIG. 5 illustrates the delivery catheter as it is configured after the plug ejection mechanism has been activated and the plug has been uncovered.

FIG. 6 shows the distal portion of the delivery catheter of FIG. 5 after the plug ejection mechanism has been activated and the plug has been uncovered.

FIG. 7 illustrates a delivery catheter with the plug ejection mechanism adapted for use with the catheter construction depicted in our prior U.S. Pat. No. 6,309,384.

FIG. 8 shows a delivery catheter with the latch release button disposed in the proximal portion of the handle.

FIG. 9 shows a solenoid-operated version of the sheath retraction system of FIG. 3 which may be used in the delivery catheters of FIGS. 1, 2, 4 and 5.

FIG. 10 illustrates a catheter body with a distal ball tip, adjacent hinge and side wall opening.

FIG. 11 illustrates the hinge and ball tip bending under pressure to prevent injury to the tissue.

### DETAILED DESCRIPTION OF THE INVENTION

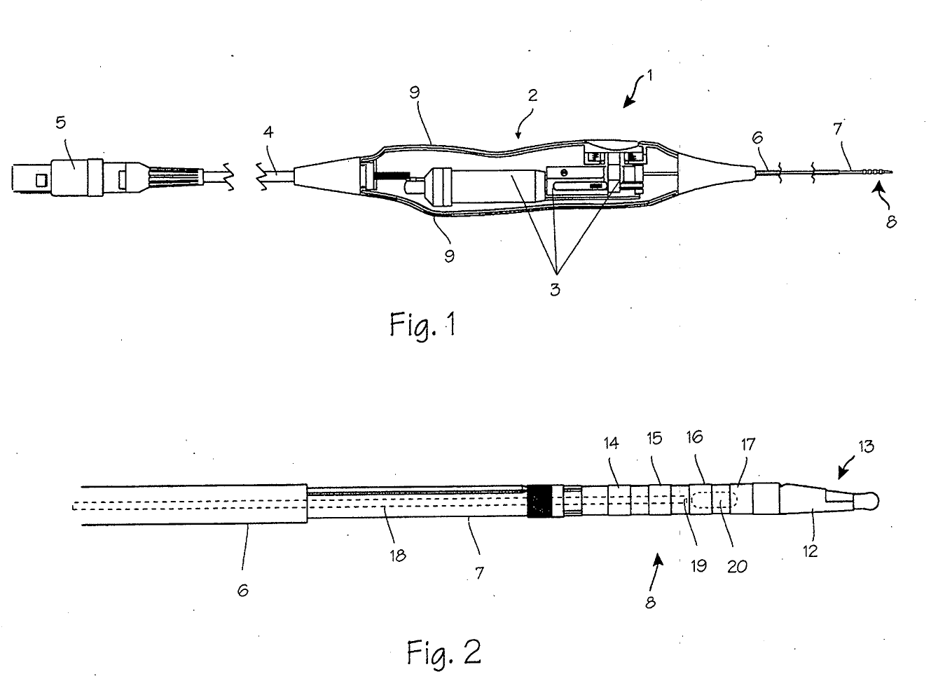

FIG. 1 is a sectional view of the delivery catheter 1 illustrating the main components of delivery catheter 1. The delivery catheter 1 comprises an ergonomic handle 2 housing a sheath retraction mechanism 3, a cable 4 with an electrical connector 5, a catheter body or shaft 6, a sheath 7 and a plug 20 (not visible in the view) disposed within the wounding segment 8. The sheath 7 comprises a tube slidably disposed within the catheter body 6. The sheath extends proximally to the handle and distally from the distal end of the catheter body 6. The handle comprises a housing 9 which contains the sheath retraction mechanism 3 and support structures for the catheter body, connectors and other components, and provides an ergonomic handgrip for the clinician. The cable and connector are suitable for connection to a power source and control system, which is operable to provide power to the wounding segment. FIG. 2 shows the distal portion of the delivery catheter, including the distal portion of the sheath which protrudes distally from the distal end of the catheter body 6. The distal portion of the sheath 7 comprises the wounding segment 8 coupled to a tapered atraumatic distal tip 12. A plug opening slit 13 is located on one side of the sheath approximately 3 mm proximal to the end of the distal tip. Electrodes 14, 15, 16 and 17 on the outer surface of the wounding segment 8 are operable to lightly wound the ovarian pathway as described in Harrington, U.S. Pat. No. 6,309,384. A push rod assembly 18 is disposed within the sheath and catheter body. The distal tip 19 of the push rod is located within the internal diameter of the sheath 7 and against the proximal end of the plug 20. The push rod assembly maintains the plug in position within the ovarian pathway while the sheath 7 and wounding segment are pulled proximally within the catheter body 6 when the plug ejection mechanism is operated.

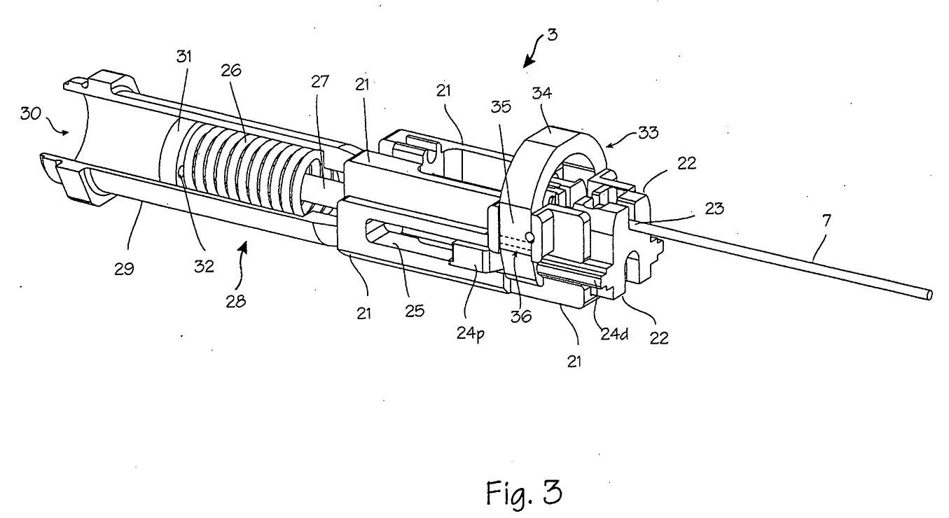

FIG. 3 shows a perspective view of the major components of the sheath retraction mechanism. The sheath retraction mechanism comprises a chassis 21 and a sliding hub 22 that is slidably engaged with the chassis. The chassis is fixedly attached to the housing of the handle shown in FIG. 1. The proximal end 23 of the sheath 7 is longitudinally fixed to the sliding hub. (The pushrod 18, hidden in this view, extends proximally beyond the proximal end of the catheter body, and is fixed to the chassis at the proximal end of the chassis.) Rail guides in the form of ridges or extensions 24d and 24p coupled to the sliding hub fit within rail slots 25 of the chassis, such that the sliding hub is slidably secured within the chassis. A spring 26 is operably coupled to the sheath through a piston rod 27 which is coupled and longitudinally fixed to the sliding hub. The spring is disposed within a dashpot 28 that comprises a cylinder 29 with a fluid-filled chamber 30. (The fluid within the chamber may be a liquid or gel having sufficient viscosity to regulate the expansion of the spring 26 and retract the sheath smoothly. Suitable fluids include materials such as silicone oil.) The spring engages a piston 31 slidably disposed within the chamber. The rod 27 is coupled to the piston at the rod's proximal end and coupled to the sliding hub at the rod's distal end. (Perforations 32 in the piston allow movement through the fluid within the chamber.) A latch 33 is used to hold the sliding hub in the distal position, and thus hold the spring in a compressed position, as shown in FIG. 3. The latch is U-shaped with a base 34 and extensions 35 that extend downward into interfering relationship with the distal rail guide 24d. The yoke is slidably disposed about the chassis and may slide up and down relative to the chassis and sliding hub. The extensions contain a channel 36 (shown in phantom) sized and dimensioned to accommodate the distal rail guides 24d. When the yoke is in an up position the yoke channel is misaligned with the rail guide, preventing proximal movement of the sliding hub. When the yoke is moved down such that the channel 36 is aligned with the rail guide, the system is unlatched and the sliding hub and sheath are pulled proximally by the expanding spring. (The components shown in this view are symmetrical about the long axis of the device, so that the hidden side of sheath retraction mechanism is identical to the illustrated view, but the device need not be symmetrical. Also, the dashpot assembly illustrated has the spring disposed within the fluid chamber of the dashpot, but the spring may be located in any other convenient position in the system (for example, proximal to the sliding hub).)

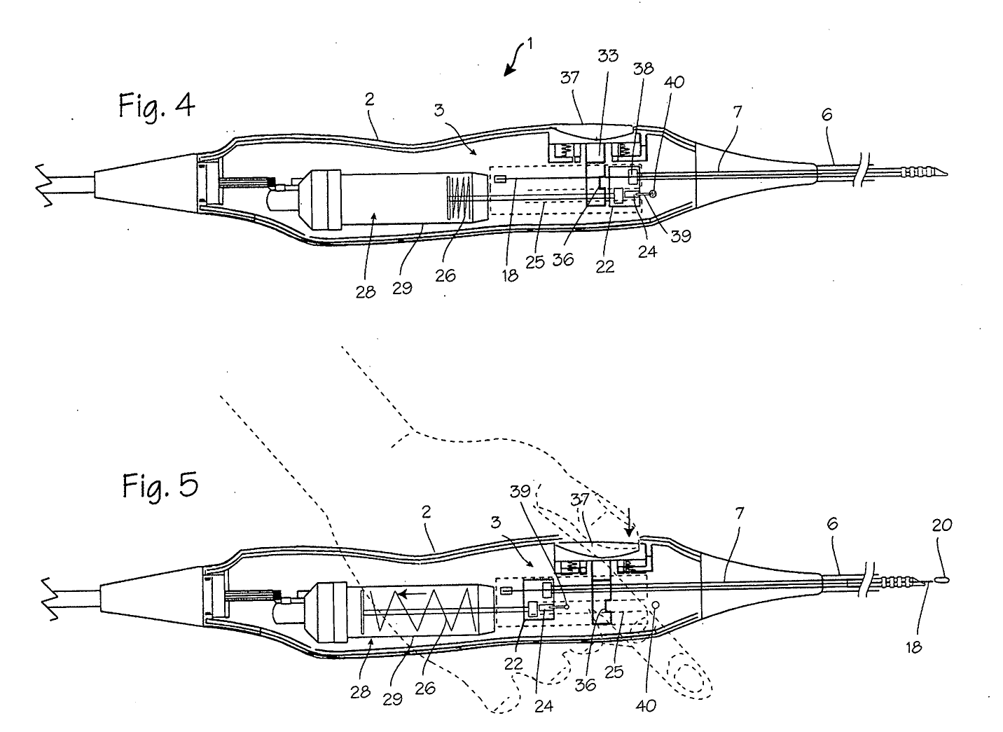

FIG. 4 illustrates the delivery catheter 1 with the plug ejection mechanism as it is configured prior to ejection of the plug. Here, the spring 26 is compressed within the cylinder 29. The sliding hub 22 is in a distal position within the handle 2. The yoke 33 is holding the sliding hub 22 in this position. A latch release button 37 is positioned above the yoke 33 in such a manner so as to impinge upon the yoke 33 when the push-button 37 is depressed. The channel 36 is misaligned with the rail guide 24 and the rail slot 25. The push rod assembly 18 is longitudinally fixed relative to the chassis 21 and handle. The sliding hub 22 is coupled to the sheath 7 at its proximal end by boss 38 (which is fixed to the proximal end of the sheath and captured within the sliding hub) but is not attached to the underlying push rod 18.

FIG. 5 shows the plug ejection mechanism after as it is configured after it has been activated and the plug 20 has been uncovered. In this view, the push-button 37 has been depressed and has moved the yoke 33 downward. When the button 37 is depressed and the yoke 33 is in a down position, the channel 36 is aligned with the rail guide 24 and the rail slot 25. Thus, the rail guide 24 is able to slide within the channel 24 along the slot 25 when the release button 37 is depressed. This allows the spring 26 to expand and force the piston, rod, sliding hub and sheath proximally relative to the chassis and handle, while the push rod 18 is held in place relative to the withdrawn sheath. Withdrawal of the sheath 7 within the catheter body 6 deposits the plug from the distal tip of the catheter without moving the plug relative to a wounded segment of the ovarian pathway after initial positioning (and also without moving the catheter body relative to the patient).

To provide feedback to the physician that the plug ejection is complete, a first contact 39 is deposed on a rail guide and a 30 second contact 40 is mounted on the wall of the handle or otherwise fixed relative to the handle and/or chassis. An electrical circuit is closed as long as the first and second contacts remain in electrical communication with one another.

Energy can be supplied to the wounding element while this electrical communication is maintained. When the sheath retraction mechanism 3 is activated, the sliding hub 22 is forced proximally (and the sheath 7 is retracted), and the first contact slides past the second contact. The first and second contacts are no longer in electrical communication with one another when the sliding block is in the proximal position within the handle. The loss of contact is sensed by the control system, which provides visual or audio indication to the clinician indicating that the sheath has been withdrawn. The control system may also be programmed such that it will not provide power to the wounding segment if contact between the two electrodes has been broken. In conjunction with the control system, which is programmed to provide appropriate interface indications and apply power only if the contacts are in electrical communication, this limits the possibility that doctor might try to insert a catheter that is not properly loaded, or which has been used or prematurely released.

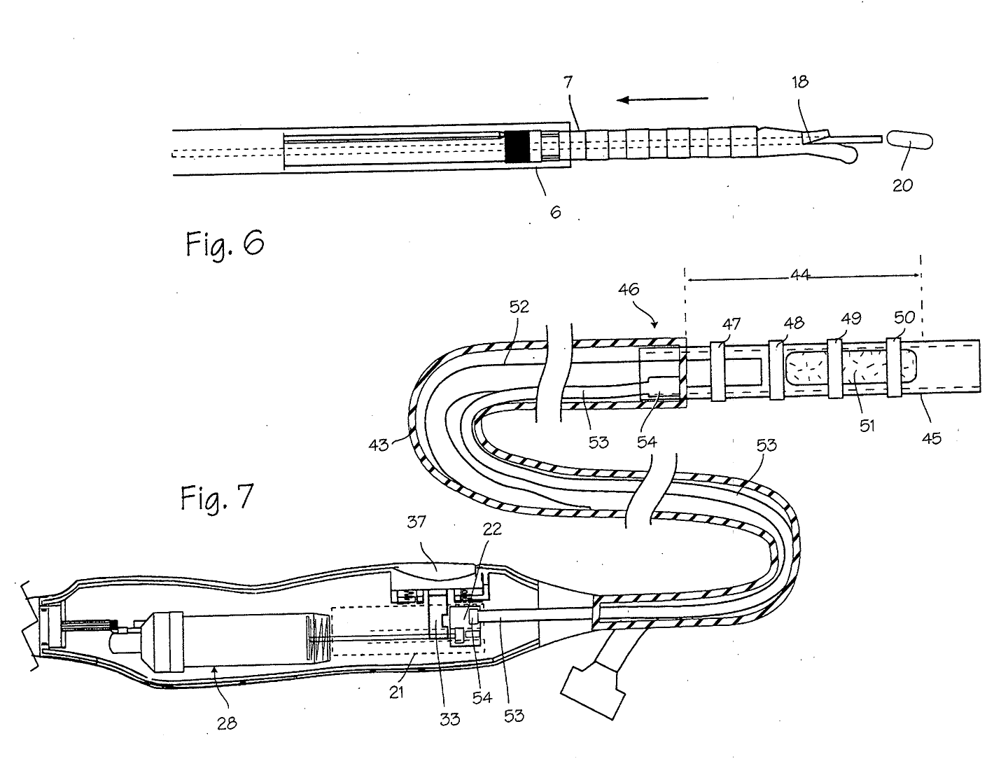

FIG. 6 shows the distal portion of the delivery catheter after the plug ejection mechanism has been activated and the plug is ejected. As illustrated in FIG. 6, the plug 20 is uncovered and released by the retraction of the sheath 7 over the plug 20 as it is held in position by the push rod 18.

FIG. 7 illustrates a delivery catheter with the plug ejection mechanism adapted for use with the catheter construction depicted in our prior U.S. Pat. No. 6,309,384. As shown in FIG. 7, the delivery catheter comprises a catheter body 43 with a wounding segment 44 comprising a short tubular sheath 45 slidably mounted within the distal tip 46 of the catheter. The distal tip of the catheter body extends over the proximal end of the tubular extension for a short length. Four electrodes 47, 48, 49 and 50 are disposed along the outer surface of the wounding segment and wrap around the catheter.

One or more foam plugs 51 are stored within the catheter body, and are shown housed within the wounding segment. A push rod 52 is disposed within the catheter body 43, fixed longitudinally within the catheter body at a point proximal to the wounding segment which permits adequate pullback of the wounding segment sheath 45 to uncover and release the plug, in contrast to the holding rod of FIGS. 1 through 5 which extends into the handle to a fixation point proximal to the proximal end of the catheter body. Unlike the sheath shown in FIGS. 1 through 5, the sheath 45 of FIG. 7 does not fully extend to the chassis 21 and is not directly coupled to the sliding hub 22. Instead, a pullwire 53 is secured to the proximal end of the sheath 45 and wounding segment by attachment of the boss 54 on the distal end of the pullwire 53. The pullwire 53 extends proximally from the wounding segment to the hub and is longitudinally fixed to the sliding hub 22. The dashpot 28, latch 33, and pushbutton 37 are arranged as described above. When the plug ejection mechanism is activated, the pullwire and the sheath 45 are retracted proximally as the sliding hub 22 slides proximally within the chassis 21. Thus, various arrangements of the translating components and fixed components of the system may be employed in the plug ejection mechanism.

In use, the clinician places the distal end of the catheter system at the appropriate location within the ovarian pathway of the patient, using appropriate visualization and manipulation the catheter with the handle. Thereafter, the clinician will operate the control system of the system to apply appropriate energy to the ovarian pathway proximate the wounding segment.

Thereafter, the clinician, holding the catheter system in one hand or both hands, need only depress the push-button to release the plug into the wounded segment of the ovarian pathway. Using the configuration described above, all necessary manipulations may be accomplished one-handed, leaving the clinician's other hand free to manipulate the control system or a hysteroscope.

If it is desired to configure the device so as to inhibit one-handed operation, the device may be configured as shown FIG. 8, which illustrates a configuration of the handle and sheath retraction mechanism with the push-button disposed in the proximal portion of the handle, thereby encouraging two-handed use of the device. The delivery catheter of FIG. 8 includes the catheter shaft or body 6, a sheath 7 and housing 9. The plug ejection mechanism 3 is contained within the handle. A release button 37 operably coupled to the plug ejection mechanism is disposed in the proximal portion 60 of the handle. The shaft retraction mechanism is rearranged, with the dashpot 28 placed distally of the sliding hub 22, and the spring disposed to push, rather than pull, the sliding hub proximally.

With this arrangement, with the housing held most conveniently, the push-button is disposed proximally of the clinician's preferred hand (the hand 61 used to manipulate the catheter), thus encouraging or requiring that the clinician use his other hand 62 to depress the push-button. This delivery catheter may require two hands to operate. The plug ejection mechanism may also be modified to use a spring that pulls on the piston rather than push against the piston in order to retract the sheath.

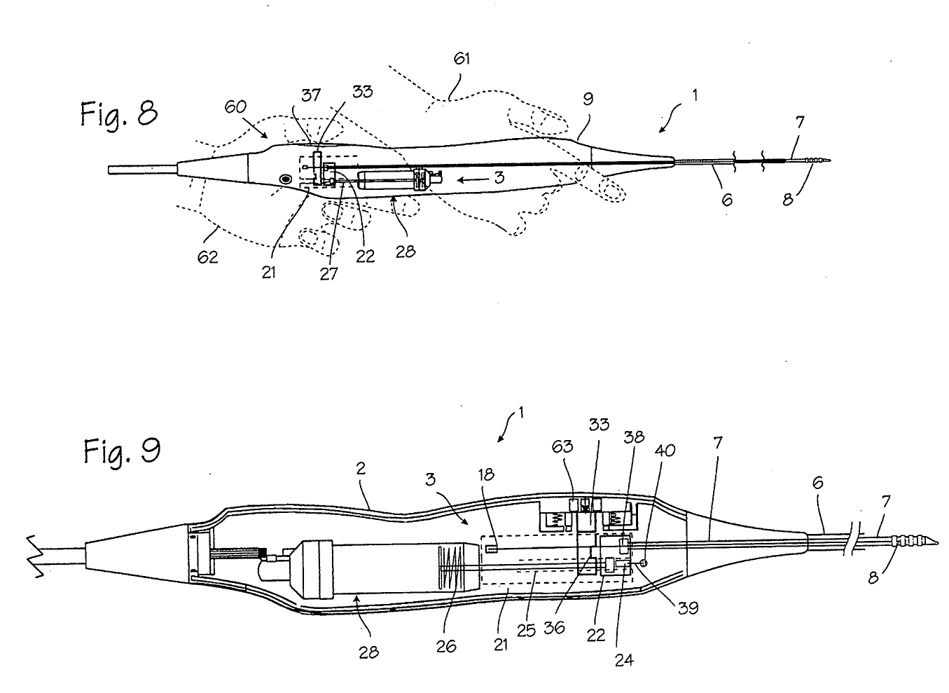

The sheath may be pushed or pulled, so long as the sheath is retracted within the body. The shaft retraction system can also be modified so that the clinician need not manually depress a push-button to force the latch downward. FIG. 9 shows a solenoid-operated version of the sheath retraction system of FIGS. 1 through 5. In FIG. 9, a solenoid 63 is operably coupled to the yoke and is used in place of a release button. Other components of the plug ejection mechanism, including the chassis 21, the sliding hub 22, the dashpot 28 and the latch 33 are arranged as shown in FIG. 4 or FIG. 8. In this device, the solenoid is placed in electrical communication with a control system. The control system is programmed to activate the plug ejection mechanism upon receipt of appropriate input from the operator, or immediately after the wounding energy has been applied. The solenoid operates to push the yoke downward to align the channels with the rail guide of the sliding hub and thereby permit proximal movement of the sliding hub. This “no-hands” configuration has the benefit that it can be controlled by the control system, and the control system can be further programmed to energize the solenoid (and eject the plug) only after the wounding segment has been operated, thereby avoiding inadvertent ejection or release without the requisite wounding steps.

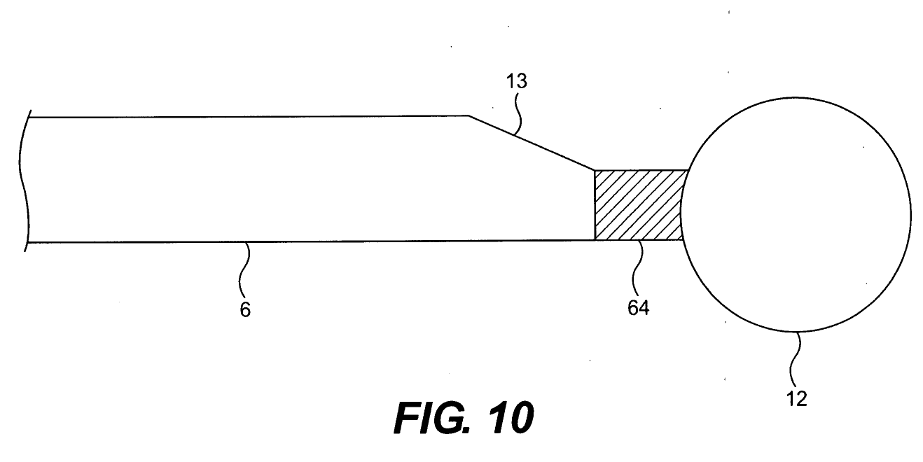

FIG. 10 illustrates a catheter body or shaft 6 with a distal ball tip 12, adjacent hinge 64 and side wall opening. In FIG. 10, the catheter body 6 has a distal end and a proximal end and an elongated portion in between the distal and proximal ends. A handle 2 is located at the proximal end of the catheter body 6. The catheter body 6 is tapered at the distal end forming the distal ball tip 12. The ball tip 12 is configured to navigate through the uterus and the fallopian tube such that the plug may be inserted into the fallopian tube to sterilize the patient. The distal ball tip 12 is spherical in shape. The distal ball tip 12 has a diameter in a range from about 0.02 inches to about 0.05 inches and more preferably from about 0.03 inches to about 0.04 inches. The ball tip 12 may be either hollow or solid but it is preferably solid.

Thermoplastic elastomers are used to form the catheter body 6 including the distal tip 12 and adjacent hinge 64. The catheter body 6 may be formed using a single thermoplastic elastomer material or different thermoplastic elastomer materials may be used to form the different portions of the catheter body 6.

Polyurethane is the preferred material to form the catheter body. Examples of other suitable materials that may be used to form the catheter body are polyvinyl chloride, polyamide, polypropylene, polyethylene, Pebax® and nylon.

The hardness of the material selected to form the catheter body 6 is an important feature of the invention. Polyurethane having a durometer in the range of about 35 to about 72 durometer on the Shore D hardness scale is preferred. Polyurethane having a durometer in the range of about 42 to about 62 on the Shore D hardness scale is most preferred.

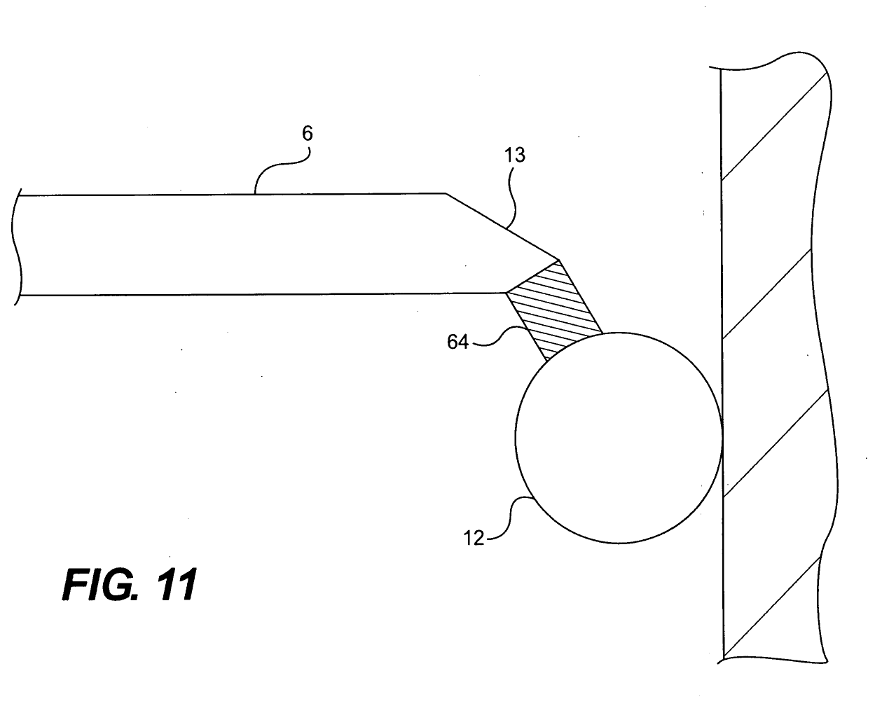

There are several different embodiments disclosed for the hinge 64. In all embodiments, however, the hinge 64 is designed to operate such that the distal ball tip 12 bends or yields under high loads to prevent the puncture of the fallopian tube or uterine wall. FIG. 11 illustrates the adjacent hinge 64 and ball tip 12 bending under pressure to prevent injury to the epithelium. If the hinge 64 does not cause the ball tip 12 to bend under pressure, the ball tip 12 may puncture the patient's epithelium during a procedure. Because the hinge 64 causes the ball tip 12 to yield, the ball tip 12 and the catheter 1 may be used to navigate through the fallopian tubes, uterus or other vessel of the body without causing any injury to the patient.

In one embodiment of the invention, the hardness of material selected to form the distal ball tip 12 is greater than that of the hardness selected for the adjacent hinge 64. For example, the hinge 64 may be composed of polyurethane having a durometer of 42 while the ball tip 12 may be composed of polyurethane having a durometer of 55. Because the hinge 64 has a softer durometer than the ball tip 12, the ball tip 12 will yield or bend at the hinge in response to a high pressure load. This prevents the ball tip 12 from puncturing or damaging the epithelium when the catheter is inserted into the uterus and the fallopian tube.

In another embodiment of the invention, the geometry of the hinge 64 is altered to comprise a groove. In this embodiment, the hinge may be made of the same material having the same durometer as the ball tip. A groove is cut around the circumference of the catheter body. The groove may be V-shaped or any other geometry suitable to achieve the purpose of the hinge. The size of the groove corresponds to the failure or bending of the hinge and ball tip under a certain pressure. For example, a larger groove or a groove that has a larger depth will yield under lower pressures. Smaller grooves or grooves that have a smaller depth will yield only under higher pressures. The groove may be formed by removing material from the catheter body after the catheter is formed at the location desired of the hinge.

Alternatively, the hinge may be formed by forming a catheter body 6 having a thinner wall at the desired location for the hinge 64. The thinner wall at the location of the hinge 64 on the catheter body 6 ensures that the hinge 64 will yield under pressure and cause the distal tip 12 to bend. The hinge 64 may also be formed by simply heat treating the catheter body 6 at the desired location for the hinge 64. Heat treating the thermoplastic elastomer material selected for the catheter body 6 will alter the yield strength at the location of the hinge 64.

The side wall opening 13 is located along the elongated catheter body 6. The implant or contraceptive plug is discharged through the side wall opening 13. An important feature of the side wall opening 13 is that it allows the implant or contraceptive plug to exit the catheter body 6 without disturbing the distal ball tip 12. In one embodiment, the side wall opening 13 is angled such that the implant exits the side wall of the catheter body 6 at the proper orientation and in a concentric fashion to occlude the fallopian tube.

The side wall opening 13 may be shaped in a variety of different ways. In one embodiment, the opening 13 may be in the shape of simple slit across the catheter body. The slit must be sized large enough for the implant or plug to be discharged through the slit. For example, an effective length for the slit may be about 3 mm. One advantage of the slit configuration for the opening is that the slit returns to a closed position after the implant is discharged and the catheter body may be used a second time. The slit opening is particularly beneficial when using the catheter body to implant occluding plugs into the fallopian tubes.

In another embodiment, the side wall opening may take the form of a flap rather than a slit. The flap may be different sizes and geometries. The size and shape of the flap must be sized to accommodate the push rod discharging the implant through the flap. For example, the flap may be configured as an M-cut made in the catheter body. A flap may be more appropriate than a slit depending on the size and shape of the plug or implant that is being used with the catheter.

The plug ejection mechanism and sheath retraction system can be adapted to deliver other contraceptive devices, occlusive devices intended for other lumens of the body, and other implants. Thus, while the preferred embodiments of the devices and methods have been described in reference to the environment in which they were developed, they are merely illustrative of the principles of the inventions. Other embodiments and configurations may be devised without departing from the spirit of the inventions and the scope of the appended claims.

What is claimed is:

1. A catheter for insertion in a fallopian tube comprising:
an elongated catheter body having a distal end and a proximal end;
a handle located at the proximal end of the catheter body;
a ball tip located at the distal end of the catheter body;
a hinge located along the catheter body adjacent to the ball tip and configured to yield under high load; and
an opening located along the catheter body proximal to the hinge.

2. The catheter of claim 1 wherein the opening is a slit.

3. The catheter of claim 1 wherein the opening is an M-cut flap.

4. The catheter of claim 1 wherein the catheter body is a thermoplastic elastomer.

5. The catheter of claim 1 wherein the hinge has a softer durometer than that of the ball tip.

6. The catheter of claim 1 wherein the hinge comprises a groove.

7. The catheter of claim 1 wherein the ball tip has a diameter in the range of about 0.02 inches to about 0.05 inches.

8. The catheter of claim 1 wherein the ball tip has a diameter in the range of about 0.03 inches to about 0.04 inches.
